(19) United States
(12) Patent Application Publication
(10) Pub. No.: US 2014/0371171 A1
(43) Pub. Date: Dec. 18, 2014

(54) Aqueous Lock Composition Comprising Ethanol and a Polysaccharide Anticoagulant

(71) Applicants: UNIVERSITE D'AUVERGNE CLERMONT I, CLERMONT FERRAND (FR); CENTRE HOSPITALIER UNIVERSITE DE CLERMONT-FD, CLERMONT FERRAND CEDEX (FR)

(72) Inventor: Bertrand Souweine, Clermont Ferrand (FR)

(21) Appl. No.: 14/371,937

(22) PCT Filed: Jan. 11, 2013
(86) PCT No.: PCT/EP2013/050437

§ 371 Date: Jul. 11, 2014

(30) Foreign Application Priority Data
Jan. 12, 2012 (FR) 1250309

(51) Int. Cl. A61L 33/08

(52) U.S. Cl. CPC A61L 33/08; A61L 2300/42
USPC 514/56

(57) ABSTRACT

The invention relates to an aqueous lock composition comprising a compound selected from the polysaccharide anti-coagulants and ethanol, and a method for locking an intravascular device implanted in a patient comprising the introduction of said composition into the device.

The invention relates to an aqueous lock composition comprising a compound selected from polysaccharide anticoagulants and ethanol. The invention relates more particularly to an aqueous lock composition having antiseptic and antimicrobial properties and intended to be used in any intravascular device, in particular in catheters implanted in a patient.

The invention also relates to a method for locking an intravascular device implanted in a patient comprising the introduction into the device of the said aqueous lock composition.

Every year, millions of central venous catheters are implanted in order to administer intravenous treatment medications, in particular anti-infective, anticancer and immunomodulatory chemotherapies, parenteral nutrition or even labile blood products. These central venous catheters may also be used for hemodynamic monitoring of patients and for the delivery of hemodialysis.

The risk of septicemia associated with catheter use represents the greatest complication by order of frequency of incidence. The malfunctioning of catheters, mainly related to thrombosis associated with these catheters are relatively frequent, causing infection thereof and thus requiring their replacement.

In order to prevent thrombosis associated with catheters when the catheters are not in use but left in place in the patient, it is a conventional approach to instill an anticoagulant in the lumen of the catheter. This is usually a solution that is heparin-, fibrinolytic-, or citrate-based. This strategy is commonly used in hemodialysis.

In spite of improved aseptic precautions during the placing and changing of dressings and bandages, the education and training of medical personnel and caregivers, the rate of infections associated with central venous catheters remains high.

Among the complementary strategies for the prevention and treatment of catheter related infections, the use of anti-infective lock compositions is becoming increasingly more widespread.

The strategy based on an anti-infective lock consists of instilling an antimicrobial composition into the catheter lumen and leaving it in place for a variable period.

There have already been studies demonstrating the effectiveness of antibiotic-based lock compositions whether or not combined with heparin, for preventing or treating infections associated with catheters in patients undergoing dialysis and in cancer patients (Vercaigne et al, Journal of Antimicrobial Chemotherapy (2002). 49, 693-696). However, the use of antibiotics exposes one to a risk of emergence of resistance.

Ethanol is an antiseptic that is powerfully bactericidal against the biofilm formed. It is known that the antiseptic activity of ethanol depends on its concentration and on the exposure time.

There has already been disclosure of a lock composition for catheters comprising a 60% ethanol solution and heparin in a concentration of 500 IU/mL, and presenting antimicrobial properties (Ackoundou-N'guessan et al, Nephrol Dial Transplant (2006); 21, 3339-33402006). However, this document does not provide any indication as to the stability of the solution. Moreover, no description has been provided of any anti coagulation activity.

There has also been disclosure in document WO 2003/063745, of an antibacterial composition that may include an antithrombotic agent such as heparin and an antibacterial agent such as ethanol.

There has also been disclosure in document WO 2004/108091, of an antimicrobial composition including at least one alcohol such as ethanol in a concentration of between 10% and 80% v/v (volume of ethanol/volume of water), an antimicrobial agent and an anticoagulating agent such as heparin.

However, WO 2003/063745 and WO 2004/108091 do not specifically disclose a composition comprising heparin and ethanol, both in very precise relative proportions.

Indeed, it is well known that concentrated ethanol is used in analytical chemistry for extracting by precipitation certain compounds and in particular mucopolysaccharides, including heparin. It is also commonly accepted that such a precipitation cancels out the anti-coagulant properties of heparin.

Thus, at the present time, there are no stable lock compositions that combine antibiofilm, anticoagulant and detergent properties, but that do not induce microbial resistance, degradation of catheters and toxicity associated with the systemic diffusion of the composition.

Thus, a first objective of the invention is to provide a lock composition that provides a solution to all or part of the problems arising with compositions according to the state of the art.

Another objective of the invention is to provide a method for reducing or even eliminating the risks of microbial infection and coagulation in an intra-vascular device, in particular a catheter implanted in a patient.

The object of the present invention relates to an aqueous lock composition comprising:
from 10 to 6,000 IU/mL of at least one compound (a) selected from polysaccharide anticoagulants, and
from 20 to 55% v/v of ethanol.

The term “polysaccharide anticoagulant” is understood to refer to any agent having a polysaccharide structure and capable of delaying, or indeed preventing the coagulation of blood.

The term “polysaccharide anticoagulant content expressed in IU/mL” is understood to refer to the polysaccharide anticoagulant content expressed in International Units per millilitre of aqueous composition.

The term “ethanol content by volume” is understood to refer to the ethanol content expressed in mL per 100 mL of aqueous composition.

According to the invention, the composition comprises from 25 to 55% v/v, preferably from 25 to 50% v/v of ethanol.

According to the invention, compound (a) may be selected from low the molecular weight heparins.

The term “low molecular weight heparin” is understood to refer to any heparin having an average molecular weight less than or equal to 12,000 Daltons, preferably ranging from 3,000 12,000 Daltons, advantageously ranging from 3,000 to 8,000 Daltons.

According to the invention, compound (a) may be selected from enoxaparin, tinzaparin, nadroparin and dalteparin and derivatives thereof.

In an advantageous manner, compound (a) is selected from a salt of enoxaparin, a salt of tinzaparin, a salt of nadroparin and a salt of dalteparin.

In terms of salts, mention may be made especially of calcium or sodium salts.

In one embodiment of the invention, the composition comprises:
from 10 to 4700 IU/mL, preferably from 10 to 600 IU/mL of a salt of nadroparin and
from 25 to 45% v/v of ethanol.

In an advantageous manner, the composition according to the invention comprises 450 IU/mL of a salt of nadroparin and 40% v/v of ethanol.

In another embodiment of the invention, the composition comprises:
from 10 to 3,200 IU/mL, preferably from 10 to 700 IU/mL of a salt of tinzaparin and
from 25 to 40% v/v of ethanol.

In an advantageous manner, the composition according to the invention comprises 100 IU/mL of a salt of tinzaparin and 40% v/v of ethanol.

In another embodiment of the invention, the composition comprises:
from 10 to 1,900 IU/mL, preferably from 10 to 1,500 IU/mL of a salt of dalteparin and
from 25 to 50% v/v of ethanol.

In an advantageous manner, the composition comprises 200 IU/mL of a salt of dalteparin and 40% v/v of ethanol.

In another embodiment of the invention, the composition comprises:
from 10 to 4,600 IU/mL of a salt of enoxaparin and
from 25 to 50% v/v of ethanol.

In an advantageous manner, the composition comprises 500 IU/mL of a salt of enoxaparin and 40% v/v of ethanol.

Also in an advantageous manner, the composition comprises 400 IU/mL of a salt of enoxaparin and 40% v/v of ethanol.

In an equally advantageous manner, the composition comprises 200 IU/mL of a salt of enoxaparin and 45% v/v of ethanol.

According to the invention, compound (a) may be selected from heparinoids.

In an advantageous manner, compound (a) is danaparoid.

In one embodiment of the invention, the composition comprises:
from 25 to 2100 IU/mL of danaparoid, and
from 25 to 45% v/v of ethanol.

In an advantageous manner, the composition comprises 225 IU/mL of danaparoid and 40% v/v of ethanol.

According to the invention, compound (a) may be selected from pentasaccharide anticoagulants.

In an advantageous manner, compound (a) is fondaparinux.

In one embodiment of the invention, the composition comprises:
from 20 to 700 IU/mL of fondaparinux, and
from 25 to 45% v/v of ethanol.

In an advantageous manner, the composition comprises 235 IU/mL of fondaparinux and 40% v/v of ethanol.

According to the invention, the composition may also include a compound selected from the different salts of compound (a) or the dispersing agents.

According to the invention, the salt may be selected from the sodium salts, such as sodium chloride, sodium gluconate or sodium bicarbonate; the magnesium salts such as magnesium chloride; the calcium salts such as calcium chloride, calcium sulfate or calcium gluconate, and the potassium salts such as potassium chloride or potassium gluconate.

In an advantageous manner, the salt is sodium chloride.

According to the invention, the dispersing agent is a dispersant, a dispersant that is stable in the presence of ethanol and biocompatible with the constituent invasive materials of intravascular devices, in particular catheters, and may be an enzyme.

In an advantageous manner, the dispersant is Dispersin B or an amylase or a nuclease.

According to the invention, the composition may be used for locking intravascular devices.

The term “intravascular device” is understood to refer in particular to intravascular catheters or implantable catheter chambers.

In an advantageous manner, the composition according to the invention is used for locking intravascular catheters.

According to the invention, the intravascular device is implanted and left in place in the patient, without being used for a period that may range from 1 day to 5 years.

An object of the present invention also relates to a kit for the preparation of an aqueous lock composition according to the invention, comprising:
an aqueous solution (b) comprising from 10 to 6,000 IU/mL of a compound (a) selected from polysaccharide anticoagulants, in particular heparinoids or heparins, notably low molecular weight heparins, and
an aqueous solution comprising from 20 to 55% v/v of ethanol (c).

The various features or preferences pertaining to the composition according to the invention presented for compound (a) are also applicable to the kit according to the invention.

According to the invention, solutions (b) and (c) are mixed extemporaneously.

According to the invention, the extemporaneous mixing of solutions (b) and (c) is carried out shortly before or at the time of its introduction into the intravascular device.

In an advantageous manner, the extemporaneous mixing of solutions (b) and (c) is carried out at the bedside of the patient.

This embodiment provides the ability to optimise the control of the stability over time of the composition, by minimising as far as possible the contact time between compound (a) and ethanol, and thereby minimising the risk of precipitation.

Another object of the invention relates to a method for locking an intravascular device, in particular a catheter, implanted in a patient, comprising the introduction into the device of a lock composition according to the invention.

According to the invention, the composition is introduced into the intravascular device in a volume ranging from 0.2 to 5 mL, preferably from 0.5 to 2.5 mL.

According to the invention, the composition may be maintained in the intravascular device for a period of time corresponding to the period elapsing between each individual administration of a product, medicament or treatment to the patient.

According to the invention, the composition may be introduced into the intravascular device simultaneously with each individual administration of a product, medicament or treatment to the patient and then maintained in the device for a period of time ranging from 1 minute to three months, preferably from 30 minutes to 3 weeks.

Another object of the invention relates to an apparatus for introducing an aqueous lock composition into an intravascular device, in particular a catheter, the apparatus comprising:
a syringe,
an aqueous lock composition according to the invention.

In an advantageous manner, the syringe is sterilised.

Another object of the invention relates to a pretreatment method for pretreating an intravascular device, in particular a catheter, comprising the impregnation of the device by means of a lock composition according to the invention.

In one embodiment, the device is soaked in the lock composition according to the invention. The device thus treated then possesses improved antimicrobial and anticoagulating properties.

The present invention and its various embodiments will be better understood upon reading the examples that follow. These examples are provided purely by way of illustration, and without any limitation whatsoever.

### EXAMPLE 1

### Study of the Stability of Aqueous Lock Compositions According to the Invention for Hemodialysis Catheters Comprising Different Concentrations of Ethanol

### Preparation of Samples:

Various mixtures of ethanol/enoxaparin sodium were prepared in 5 mL glass hemolysis tubes combining either:
a) a fixed concentration of enoxaparin sodium with 400 IU/mL (corresponding to the dose used in an antithrombotic preventive lock) in a range of increasing concentrations of ethanol (30%, 40%, 45%, 50%, 60% and 70% by volume (v/v)), the dilutions of ethanol having been made in physiological serum solution (NaCl in a concentration of 0.9 g/100 ml).
b) increasing concentrations of enoxaparin sodium (50, 100, 400, 500 and 1,000 IU/mL) in an ethanol solution of fixed concentration (40, 45 or 50% v/v).

### Visual Observations:

The visual observation of different ethanol/enoxaparin mixtures was carried out by way of an assessment of the presence (or absence) of precipitation at the time of effecting the contacting of the two solutions; the observed phenomena corresponding, either to the absence (level 0 noted −), or the presence of a precipitation.

The extent of the precipitation was assessed, either based on the appearance of a cloudiness, or on the formation of a deposit in the form of droplets and the levels were evaluated by the scores: +, ++, +++. With respect to the assessment of the formation of a deposit, a first level corresponding to the formation of a film was added and the scores +, ++, +++ were used for the formation of droplets.

The observation was monitored over time, that is after 1 minute, 1 hour, 24 hours, 48 hours and 72 hours, of contact, and this was done at two temperatures, 20° C. and 37° C.

### Assay for Determination of Ethanol

The stability of the ethanol was monitored and controlled by means of an assay according to the method of Nicloux (Traité d'œnologie sciences et techniques du vin/Treatise on Enology Science and Technology of Wine, Volume 1. Ribéreau-Gayon et al., published by Bordas éditeur, Paris 1976), through oxidation of ethanol by making use of a known excess of potassium dichromate, followed by a back titration of dichromate with a solution of ferrous sulfate in the presence of ferrous orthophenantroline.

The assay was performed in parallel with a blank test and, in order to overcome errors due to possible oxidation of the heparin, a prior distillation was carried out in order to selectively assay the ethanol.

### Assay for Determination of Enoxaparin

The assaying of enoxaparin was performed by a method of coupling high-performance liquid chromatography (HPLC) with an evaporative light scattering detector (ELSD) method. In order to be within the zone of linearity of the detector response, the samples were diluted to one tenth in the physiological serum solution. The analysis was carried out in the mixtures combining enoxaparin sodium 400 IU/mL with increasing concentrations of ethanol (30 to 70% (V/V)), in comparison with a calibration range of the enoxaparin composition (marketed under the trade name Lovenox®) prepared in the physiological serum solution at the following concentrations: 6 IU/mL, 12 IU/mL, 40 IU/mL, 80 IU/mL, 120 IU/mL.

For the samples presenting a cloudiness effect, a preliminary centrifugation at 1,600 g for 5 minutes was carried out and only the supernatant was subsequently analysed.

The operating conditions for the chromatographic separation were as follows:
analytical column measuring 150 mm in length and 4.6 mm in diameter, filled with a stationary phase of C18 5 μm.
separation of components in isocratic mode making use of a mixture of methanol/water, 50/50 (V/V) at a flow rate of 0.3 mL/min.
detection using an evaporative light scattering detector (ELSD): evaporation temperature of 40° C., gain of the detector=3 and pressure of nitrogen (nebulizer gas)=3.5 bar.

### Results

### Visual Observations

The phenomena observed during the placing together in each other's presence of a composition of enoxaparin sodium 400 IU/mL and increasing concentrations of ethanol are as follows:
for ethanol concentrations less than or equal to 40% v/v, the mixture remains clear and colourless up to 72 hours at 20° C. or at 37° C.,
for higher levels of ethanol content (60 and 70% v/v), the cloudiness increases progressively as the ethanol content increases.

### Assay for Determination of Ethanol

The chemical assay method for the determination of ethanol used is intended for the routine determination of blood alcohol levels and therefore for the determination of much lower levels than those used in the lock compositions. Thus, the samples had to be diluted to 1/100 that could eventually introduce a possible additional source of error having a slight impact on the precision of the assay (calculated coefficient of variation (CV of 2.5% on average instead of the 2.0% of the validation parameters).

The combination of enoxaparin sodium 400 IU/mL and 40% ethanol (v/v) has no significant influence on the initial ethanol content.

### Assay of Enoxaparin Sodium by Means of HPLC-ELSD:

The particularity of the detection method using ELSD is that the relationship between the area of the chromatographic peak A and the mass m is rarely linear; it is usually of the type A=am^{b}, where a and b are coefficients that are dependent in particular, on the size of the droplets formed, on the nature, on the concentration and the volatility of the solute, and on the flow rates of the gas and mobile phase. A log-log representation may sometimes prove to be necessary to ensure a proper calibration.

It turned out in the present application that the area of the chromatographic peak A of enoxaparin was not directly proportional to the mass m and that a linearity in the form of Y=aX+b was obtained with Y=log A and X=enoxaparin concentration in IU/mL.

The results show that for ethanol in a concentration of 70% v/v, the enoxaparin content levels found in the supernatant after centrifugation are not detectable. The enoxaparin is practically no longer soluble and has then precipitated to form droplets by decantation.

These results show that the solubility of low molecular weight heparin decreases notably for a high ethanol content, in particular equal to 70% v/v.

### EXAMPLE 2

### Study of the Stability of Aqueous Lock Compositions According to the Invention Comprising Low Molecular Weight Heparins Varying in Nature and in Different Concentrations

The lock compositions tested were as follows:
composition comprising a mixture of the nadroparin calcium and ethanol
composition comprising a mixture of the tinzaparin sodium and ethanol,
composition comprising a mixture of the dalteparin sodium and ethanol,
composition comprising a mixture of the enoxaparin sodium and ethanol.

The concentration of ethanol expressed in % of ethanol v/v that does not cause the lock composition to precipitate, was determined for each lock composition, by varying the heparin content.

The compositions were tested at ambient temperature and at 37° C. and the results were observed after 48 hours.

The results are presented in the Tables 1 to 4.

TABLE 1

| Nadroparin Calcium | Ethanol content (% v/v) | Ethanol content (% v/v) |
|---|---|---|
| content (IU/mL) | at ambient temperature | at 37° C. |
| 10 | 43.7 | 44.6 |
| 500 | 41.7 | 41.7 |
| 1,000 | 39.7 | 38.7 |

TABLE 2

| Tinzaparin | | |
|---|---|---|
| Sodium content | Ethanol content (% v/v) | Ethanol content (% v/v) |
| (IU/mL) | at ambient temperature | at 37° C. |
| 10 | 42.1 | 44.0 |
| 500 | 34.3 | 41.0 |
| 1,000 | 26.3 | 38.0 |

TABLE 3

| Dalteparin Sodium | Ethanol content (% v/v) | Ethanol content (% v/v) |
|---|---|---|
| content (IU/mL) | at ambient temperature | at 37° C. |
| 10 | 44.6 | 47.8 |
| 500 | 35.7 | 41.9 |
| 1,000 | 26.7 | 35.9 |

TABLE 4

| Enoxaparin Sodium | Ethanol content (% v/v) | Ethanol content (% v/v) |
|---|---|---|
| content (IU/mL) | at ambient temperature | at 37° C. |
| 10 | 47.6 | 48.2 |
| 500 | 40.7 | 45.8 |
| 1,000 | 33.7 | 43.3 |

The results confirm that, for a broad range of low molecular weight heparin content and for an ethanol content ranging from 20 to 50%, the aqueous lock compositions according to the invention are stable over time.

### EXAMPLE 3

### Study of the Anticoagulant Effect of a Lock Composition According to the Invention Comprising a Mixture of Enoxaparin Sodium 400 IU/mL and Ethanol 40% v/v

The anticoagulant effect of a lock composition according to the invention comprising a mixture of enoxaparin sodium 400 IU/mL and ethanol 40% v/v was evaluated in laboratory.

In this respect, two methods were used:
overall assessment of the anticoagulant effect, by measuring the thrombin generation time (TGT) in Thrombinography on platelet rich plasma (PRP);
measurement of anti-Xa activity on platelet poor plasma (PPP).

In order to closely approximate the conditions of use in vivo of the lock composition according to the invention, the following were added to the citrated blood of healthy volunteers in different quantities:
the lock composition according to the invention,
a composition of enoxaparin sodium 400 IU/mL (control).

These solutions had been prepared on the preceding day and stored at 4° C. prior to use. After incubation of the mixtures for 1 hour at 37° C., the thrombin generation time (TGT) in the PRP and the anti-Xa activity in the platelet poor plasma (PPP) were measured.

The results of the measurement of thrombin generation time (TGT) show that a dose dependent anticoagulant effect of the lock composition according to the invention was observed. The latter is close to the anticoagulant effect obtained with the composition of enoxaparin 400 IU/mL alone.

The half maximal inhibitory concentration—IC₅₀ ETP values of the lock composition according to the invention corresponding to the dose that inhibits 50% of the ETP and therefore corresponding to a satisfactory anticoagulant activity (the ETP being the endogenous thrombin potential, ie the reflection of the coagulating action of the PRP tested) are obtained for a median anti-Xa activity of 1.78 [1.05-2].

The IC₅₀ ETP of the composition of enoxaparin 400 IU/mL alone is obtained for a median anti-Xa activity of 0.68 [0.25-0.90].

The results of the measurement of the anti-Xa activity in platelet poor plasma (PPP) show that a dose dependent anticoagulant effect for the lock composition according to the invention was observed. The latter is close to the effect obtained with the composition of enoxaparin sodium 400 IU/mL alone.

The ratios (enoxaparin sodium 400 IU/mL/mixture of enoxaparin sodium 400 IU/mL-ethanol 40%) ranged between 1.5 and 2 depending on the volunteers.

Thus, for a final concentration of enoxaparin sodium 2 IU/mL in whole blood, the anti-Xa activity measured in PPP is 3.58 at a median [3.02-4.28] for the composition of enoxaparin alone against 2.38 [1.89-2.82] for the lock composition according to the invention.

Furthermore, the stability of the anti-coagulant effect of the lock composition according to the invention was observed to be maintained for 9 days.

A loss of 17% of anti-Xa activity was observed in the ethanol solvent at 24 hours. After this loss of activity, no further loss up to day 9 was observed.

These results show that a lock composition according to the invention has a satisfactory and sufficient anticoagulant activity in the envisaged conditions of use, i.e. used alone.

### EXAMPLE 4

### Study of the Anti-Biofilm Effect of a Lock Composition According to the Invention Comprising a Mixture of Enoxaparin Sodium 400 IU/mL and Ethanol 40% v/v

This involved determining the anti-biofilm activity of a composition according to the invention by measuring the number of viable bacteria (Colony Forming Unit or CFU) after 4, 24 and 48 hours of contact with the said solution.

### Microbial Strains and Culture Conditions

Strains of Staphylococcus epidermidis CIP 68.21, Staphylococcus aureus CIP 65.25 (resistant to methicillin), Pseudomonas aeruginosa ATCC 27853 and Klebsiella pneumoniae LM 21 were selected.

These bacterial strains were cultivated in a Luria Bertani medium (LB). The organisms were maintained at 70° C. in their respective medium in the presence of 15% glycerol, and for each test the biofilm was established from the original stock.

### Formation of the Biofilm in Polyurethane Catheters

Formation of the biofilm in polyurethane catheters was carried out in 60 mL aerated microfermentors as described by Ghigo (Ghigo J M, Nature (2001) 412 (6845), 442-445) as well as in the web document of the Institut Pasteur/Pasteur Institute (www.pasteur.fr/recherche/unites/Ggb/matmet.html).

Segments measuring 1 cm length of sterile polyurethane catheters (DualCath®, Medcomp, Harleyville, USA; Hemotech, Ramonville, France) were attached to the surface of the internal removable glass slide of the microfermentors.

The strains derived from the stock frozen at −70° C. were cultivated in LB medium with 0.4% glycerol for an entire night. An inoculum consisting of 10⁹ bacilli or 10⁸ cocci was used to inoculate the microfermentors comprising the segments of catheters. A continuous flow of 10 mL/h of an LB medium with 0.4% glycerol and constant aeration with sterile pressurised air (0.3 bar) were used to obtain optimal culture conditions.

After 24 hours of incubation, the catheter segments were removed from the incubator.

The biofilms formed on the catheter segments were resuspended in 5 ml of LB by using ultrasound and a vortex.

The determined volumes of dilutions of the suspensions obtained were then placed on the surface of LB nutrient agar plates in order to determine the number of viable cells (CFU) after incubation for one night at 37° C.

The number of bacteria was expressed in the common logarithm i.e. to the base 10 (log 10); the detection limit under the experimental conditions mentioned here above was 1.6 log 10 (40 CFU) per catheter segment.

### Protocol for Data Processing

The anti-biofilm activity of the lock composition according to the invention was determined on 24 hour biofilms. After incubation, the catheter segments having a biofilm were recovered, rinsed with 1 mL of saline solution and then placed in a tube containing 1 mL of each lock composition tested:
aqueous solution of ethanol in a concentration of 40% v/v, prepared from 95% ethanol diluted in a solution of sodium chloride;
aqueous composition of enoxaparin sodium 400 IU/mL;
lock composition according to the invention,
saline solution.

For each organism, the experiments were repeated three or four times, and during each test, the catheter segments were exposed to the various different solutions for 48 hours at 37° C.

Thereafter, the catheter segments were recovered, rinsed with a saline solution and the number of viable cells was determined as described here above. In addition, the biomass of the biofilm was determined prior to each treatment for each strain three times over. The catheter segments were exposed to the various solutions for 4, 24 and 48 hours.

The results are presented in Tables 5 to 8.

TABLE 5
Staphylococcus epidermidis CIP 68.21

| | | Duration of | |
|---|---|---|---|
| | | the Treatment | |
| Composition | 4 hrs | 24 hrs | 48 hrs |
| Saline solution | 2.66E+06 | 3.88E+06 | 1.65E+06 |
| Aqueous composition of | 3.43E+06 | 6.67E+06 | 1.47E+06 |
| enoxaparin sodium 400 IU/mL, | | | |
| Aqueous solution of ethanol in | ND | ND | ND |
| concentration of 40% v/v | | | |
| Lock Composition according to | ND | ND | ND |
| the invention | | | |

ND: value not determined due to it being below the detection limit

TABLE 6
Staphylococcus aureus CIP 65.25

| | | Duration of | |
|---|---|---|---|
| | | the Treatment | |
| Composition | 4 hrs | 24 hrs | 48 hrs |
| Saline solution | 5.79 + 06 | 2.29E+06 | 2.39E+06 |
| Aqueous composition of | 3.93 + 06 | 3.13E+06 | 1.84E+06 |
| enoxaparin sodium 400 IU/mL, | | | |
| Aqueous solution of ethanol in | 2.78E+04 | ND | ND |
| concentration of 40% v/v | | | |
| Lock Composition according to | 1.61E+05 | ND | ND |
| the invention | | | |

ND: value not determined due to it being below the detection limit

TABLE 7
Pseudomonas aeruginosa ATCC 27853

| | | Duration of | |
|---|---|---|---|
| | | the Treatment | |
| Composition | 4 hrs | 24 hrs | 48 hrs |
| Saline solution | 4.72E+08 | 6.61E+08 | 2.03E+08 |
| Aqueous composition of | 9.52E+08 | 7.80E+08 | 2.03E+08 |
| enoxaparin sodium 400 IU/mL, | | | |
| Aqueous solution of ethanol in | ND | ND | ND |
| concentration of 40% v/v | | | |
| Lock Composition according to | ND | ND | ND |
| the invention | | | |

ND: value not determined due to it being below the detection limit

TABLE 8
Klebsiella pneumoniae LM 21

| | | Duration of | |
|---|---|---|---|
| | | the Treatment | |
| Composition | 4 hrs | 24 hrs | 48 hrs |
| Saline solution | 4.52E+08 | 3.15E+08 | 1.13E+08 |
| Aqueous composition of | 8.12E+08 | 2.80E+08 | 1.48E+08 |
| enoxaparin sodium 400 IU/mL, | | | |
| Aqueous solution of ethanol in | 2.51E+06 | ND | ND |
| concentration of 40% v/v | | | |
| Lock Composition according to | 1.87 + 06 | ND | ND |
| the invention | | | |

ND: value not determined due to it being below the detection limit

The results show no significant difference after treatment of the biofilms between a solution of 40% v/v ethanol alone and a lock composition according to the invention, and this is so whichever bacterial strain is tested. The bactericidal activity of the ethanol is thus not altered by the presence of enoxaparin sodium 400 IU.

The results show in particular that for the two bacterial species, (i.e. Staphylococcus epidermidis and Pseudomonas aeruginosa), the eradication is maximal (below the detection limit of 40 CFU) starting from incubation time of 4 hours onwards.

For the other two bacterial species, complete eradication was observed with the incubation time of 24 hours.

These results demonstrate that a lock composition according to the invention is not only characterised by a bactericidal activity that is equivalent to ethanol, but also that this activity becomes effective rapidly, in particular starting from the initial hours after effecting the contacting with the bacterial strain, and does not exceed 24 hours.

1. An aqueous lock composition comprising:
from 10 to 6,000 IU/mL of at least one compound (a) selected from polysaccharide anticoagulants, and
from 20 to 55% v/v of ethanol.

2. A composition according to claim 1 comprising from 25 to 55% v/v, of ethanol.

3. A composition according to claim 1, wherein compound (a) is selected from low molecular weight heparins.

4. A composition according to claim 1 wherein compound (a) is selected from enoxaparin, tinzaparin, nadroparin and dalteparin and derivatives thereof.

5. A composition according to claim 1 wherein compound (a) is selected from a salt of enoxaparin, a salt of tinzaparin, a salt of nadroparin and a salt of dalteparin.

6. A composition according to claim 1 comprising:
from 10 to 4,700 IU/mL, of a salt of nadroparin and
from 25 to 45% v/v of ethanol.

7. A composition according to claim 1 comprising 450 IU/mL of a salt of nadroparin and 40% v/v of ethanol.

8. A composition according to claim 1 comprising:
from 10 to 3,200 IU/mL, of a salt of tinzaparin and
from 25 to 40% v/v of ethanol.

9. A composition according to claim 1 comprising 100 IU/mL of a salt of tinzaparin and 40% v/v of ethanol.

10. A composition according claim 1 comprising:
from 10 to 1,900 IU/mL, of a salt of dalteparin and
from 25 to 50% v/v of ethanol.

11. A composition according to claim 1 comprising 200 IU/mL of a salt of dalteparin and 40% v/v of ethanol.

12. A composition according to claim 1 comprising:
from 10 to 4,600 IU/mL of a salt of enoxaparin and
from 25 to 50% v/v of ethanol.

13. A composition according to claim 1 comprising:
500 IU/mL of a salt of enoxaparin and 40% v/v of ethanol, or
400 IU/mL of a salt of enoxaparin and 40% v/v of ethanol, or
200 IU/mL of a salt of enoxaparin and 45% v/v of ethanol.

14. A composition according to claim 1 wherein compound (a) is selected from heparinoids.

15. A composition according to claim 1, comprising:
from 25 to 2,100 IU/mL of danaparoid, and
from 25 to 45% v/v of ethanol.

16. A composition according to claim 1 comprising 225 IU/mL of danaparoid and 40% v/v of ethanol.

17. A composition according to claim 1 wherein compound (a) is selected from pentasaccharide anticoagulants.

18. A composition according to claim 1 comprising:
from 20 to 700 IU/mL of fondaparinux and
from 25 to 45% v/v of ethanol.

19. A composition according to claim 1 comprising 235 IU/mL of fondaparinux and 40% v/v of ethanol.

20. A method for locking an intravascular device comprising the use of a composition according to claim 1.

21. A kit for the preparation of an aqueous lock composition according to claim 1, comprising:
an aqueous solution (b) comprising from 10 to 6,000 IU/mL of a compound (a) selected from polysaccharide anticoagulants, and
an aqueous solution comprising from 20 to 55% v/v of ethanol (c).

22. A composition according to claim 1 comprising from 25 to 50% v/v of ethanol.

23. A composition according to claim 1 comprising:
from 10 to 600 IU/mL of a salt of nadroparin and
from 25 to 45% v/v of ethanol.

24. A composition according to claim 1 comprising:
from 10 to 700 IU/mL of a salt of tinzaparin and
from 25 to 40% v/v of ethanol.

25. A composition according to claim 1 comprising:
from 10 to 1,500 IU/mL of a salt of dalteparin and
from 25 to 50% v/v of ethanol.

26. A composition according to claim 1 wherein compound (a) is danaparoid.

27. A composition according to claim 1 wherein compound (a) is fondaparinux.

28. A method for locking a catheter comprising the use of a composition according to claim 1.

29. A kit for the preparation of an aqueous lock composition according to claim 1, comprising:
an aqueous solution (b) comprising from 10 to 6,000 IU/mL of a compound (a) selected from heparinoids and heparins, and
an aqueous solution comprising from 20 to 55% v/v of ethanol (c).

30. A kit for the preparation of an aqueous lock composition according to claim 1, comprising:
an aqueous solution (b) comprising from 10 to 6,000 IU/mL of a compound (a) selected from low molecular weight heparins, and
an aqueous solution comprising from 20 to 55% v/v of ethanol (c).
